(19) United States
(12) Patent Application Publication
(10) Pub. No.: US 2006/0253183 A1
(43) Pub. Date: Nov. 9, 2006

(54) Intramural needle-tipped surgical device

(76) Inventors: Aravinda Thagalingam, Birchgrove (AU); Pramesh Kovoor, Se Ives (AU); David Leslie Ross, Cheltenham (AU)

Correspondence Address: Woodard Emhardt, Bank One Center/Tower, 111 Monument Circle, Suite 3700, Indianapolis, IN, 46204-5137, US

(21) Appl. No.: 10/535,527

(22) PCT Filed: Nov. 7, 2003
(86) PCT No.: PCT/AU03/01482

§ 371 Date: May 26, 2006

(30) Foreign Application Priority Data
Nov. 14, 2002 (AU) 2002952663

(51) Int. Cl. A61N 1/00

(52) U.S. Cl. 607/120; 607/126

(57) ABSTRACT

Surgical devices (60) are described that are capable of creating deep thermal ablation lesions using a percutaneous or endoscopic technique. In one embodiment, the device (600) has a catheter-like member (620) with a lumen that comprises an intramural ablation needle (640) and a helical fixing member (630). The helical fixing member (630) is preferably 3 mm long and is used to fix the catheter (620) to the tissue to be ablated (610) enabling the intramural needle-like member (640) to be pushed into the tissue (610). The intramural needle-like member (640) may comprise a thermocouple to allow temperature-controlled ablation and an irrigation tube to allow irrigated needle ablation.

### FIELD OF THE INVENTION

The present invention relates generally to medical equipment and procedures, and more particularly to catheter-type devices for treating the heart and other organs.

### BACKGROUND

Radiofrequency (RF) ablation can successfully treat many cardiac arrhythmias. Unfortunately, the depth of ablation lesions produced by conventional radiofrequency ablation is limited to 4-6 mm. Active cooling of the ablation electrode has been introduced in an attempt to increase lesion depth, but even ablation lesions produced by irrigated tip catheters may not be of sufficient depth to treat the critical site of some arrhythmias. Subsequent pathological examination of patients who had unsuccessful radiofrequency ablation for intractable ventricular tachycardia revealed that the conventional, endocardial radiofrequency ablation lesion had not been transmural: e.g., see Palma, E. C. Saxenberg V, Vijayaraman P, Ferrick K, Gross J, Kim S, Fisher J, “Histopathological correlation of ablation lesions guided by noncontact mapping in a patient with peripartum cardiomyopathy and ventricular tachycardia”, Pacing Clin Electrophysiol, 24, 12, pp. 1812-5.

Radiofrequency ablation delivered through an intramural needle has been investigated as a possible means of producing true transmural ablation lesions: e.g., see Woo E J, Tungitkusolmun S, Cao H, Tsai J Z, Webster J G, Vorperian V R, Will J A, “A new catheter design using needle electrode for subendocardial RF ablation of ventricular muscles: finite element analysis and in vitro experiments,” IEEE Trans Biomed Eng. 2000, 47, pp., 23-31; and Kovoor P., “Radiofrequency ablation for ventricular tachycardia,” Department of Medicine, Sydney, University of Sydney, 1997, p. 289 and Ohtake H, Misaki T, Matsumaga Y, Watanabe G, Takahashi M, Matsumoto I, Kwawasuji M, Watanabe Y, “Development of a new intraoperative radiofrequency ablation technique using a needle electrode”, Ann Thorac Surg, 58, 3, pp. 750-3.

Most needle ablation systems are designed for use during ablation, exposing the patient to the risks and discomfort associated with major cardiac surgery.

U.S. Pat. No. 5,281,218 issued to Imran on 25 Jan. 1994 describes a catheter having a needle electrode for RF ablation of human myocardium. The catheter is an elongate member with a lumen through the catheter lengthwise. A needle electrode is rigidly fixed to a terminal end of the catheter and is connected by a conductor passing through the lumen to an RF energy source. This U.S. patent also describes a further needle electrode disposed within the catheter that can be extended from and retracted into the catheter. A significant disadvantage of such catheter-based devices is that the needle electrode often cannot be positioned so that the catheter and hence the needle electrode are relatively perpendicular to the myocardium for insertion of the needle electrode into the tissue. Instead, the catheter and hence the needle electrode often contacts the myocardium at an acute or oblique angle. Firstly, the electrode for ablation may not be positioned at the desired location and because of the angle may slide along the surface. Further, if the needle electrode enters at an acute angle, the resulting lesion produced by RF ablation may not be sufficiently deep and may instead produce a longer, but shallower lesion. Thus, healthy tissue may be destroyed needlessly. Another disadvantage of this system is that any needle electrode of sufficient width to create a clinically useful ablation lesion (>4 mm width) requires considerable force to insert the needle into the myocardium. A catheter-based system is not able to deliver that force unless the catheter is fixed firmly to the myocardium. As shown in FIGS. 7A-7C, such a catheter-based system 700 has the disadvantage of the catheter tip 710 being forced away from the myocardium 730 during attempted insertion of the needle 720. FIG. 7A illustrates the catheter device 700 ideally deployed with the catheter end 710 abutting the myocardial tissue 730; FIG. 7B shows a partial deployment of the needle 720 from the catheter; and FIG. 7C shows how the catheter end 710 moves away from the myocardium 730 when an attempt is made to deploy the needle 720.

U.S. Pat. No. 5,807,395 issued to Mulier et al on 15 Sep. 1998 and International (PCT) Patent Publication No. WO 96/07360 published 14^{th} Mar. 1996 describe methods and apparatuses for RF ablation and hyperthermia using a hollow needle electrode or helical electrode connected to a catheter to infuse conductive solution into the tissue to produce a larger virtual electrode and hence a larger treated area. A conductive fluid such as saline, saturated saline, or Ringer's solution is passed through the lumen of the catheter and is delivered via a port in the electrode at the end of the catheter into the tissue. In particular, this U.S. patent describes using a helical electrode for cardiac ablation. The helical electrode is screwed into the heart tissue by rotating the catheter body. That is, the helical electrode is screwed in and completely located within the tissue. The conductive solution is delivered via an opening at the end of the hollow electrode or via ports along the sides of the electrode. This U.S. patent discloses screwing large electrodes into the tissue of depths from 5 mm to 15 mm, that is, deeply into the myocardial tissue. However, several significant disadvantages exist in this regard. Firstly, this catheter-based device has all of the disadvantages noted above in relation to U.S. Pat. No. 5,281,218 regarding positioning of the catheter and the angle of attack of the electrode. That is, the electrode may not enter the tissue perpendicular to the surface of the myocardial tissue. Further, the helical electrode can improperly damage or destroy substantially more tissue than is the case of a needle electrode in similar circumstances if the helical electrode is pulled from the myocardial tissue and rips away more tissue in the coils at depths of up to 15 mm. For example, this might result from defibrillating the patient with the electrode in situ during a procedure. This would cause severe complications including cardiorespiratory arrest due to bleeding into the pericardial space.

U.S. Pat. No. 6,251,121 issued to Saadat on 26 Jun. 2001 describes apparatuses and methods for intraoperatively performing surgery to create transmural channels in tissue and in particular transmyocardial revascularisation. One apparatus described is a handheld device that includes a flexible hose with a cutting head coupled to a radiofrequency current source that uses low-pressure suction to stabilize an end region of the apparatus against tissue. This is done in an attempt to stabilize an end region of the device against a beating heart. Another stabilizing means comprises a corkscrew element disposed in a tubular member. The corkscrew element may be located on the distal end of the shaft adjacent to the cutting head to pierce the epicardium and urge the cutting head in contact with the heart during the channel forming process. The corkcrew element is arranged adjacent to the cutting element. This increased distance of the fixation element from the cutting element means that the fixation element may damage heart tissue outside of the region to be treated. Where the instrument is to be used as a hand held instrument held against the epicardial surface of the heart under direct vision the operator can ensure that the fixation corkscrew element is not being screwed into a vulnerable part of the heart such as coronary artery. If this same fixation technique were used in a percutaneous setting however the operator would not have direct vision of the region surrounding the area to be treated and therefore could be unsafe. U.S. Pat. No. 6,251,121 also describes a method of stabilising the handheld instrument using a plurality of resilient curved wires that diverge radially outward from the distal tip of the instrument. This technique again has the disadvantage of potentially damaging structures that are outside of the treatment zone and hence may be appropriate for use in a hand held instrument under direct vision, but not as a method for fixing a percutaneously deployed catheter.

U.S. Pat. Nos. 5,447,533 and 5,531,780 issued to Vachon et al and Vachon on 5 Sep. 1995 and 2 Jul. 1996, respectively, describe a pacing lead having a stylet-introduced, anti-inflammatory drug delivery element that is advanceable from a distal tip electrode. The drug delivery element may serve to center an active fixation element, i.e. a helix, for active fixation of the lead in the myocardium. The pacing lead is described as including an advanceable helix or corkscrew type active fixation means. The helix is usually retracted within the distal tip of the pacing lead, but can be extended from the distal tip of th pacing lead by pushing on a stylet. The user can screw the helix into myocardium by rotating the lead until the lead comes into contact with the myocardium. A dart capable of penetrating the myocardial wall is extended beyond the helix tip into the myocardium. The dart delivers therapeutic drugs to the area near the implanted tip of the helical electrode. While the helix in this configuration may be suitable for a pacing lead that does not need to be as accurately positioned within the heart chamber, this configuration is not satisfactory for an ablation catheter. During an ablation procedure, the catheter has to be carefully manipulated to a specific location in the heart, further rotation of the catheter is disadvantageous as this would displace the catheter from this location.

International (PCT) Patent Publication No. WO 99/22658 published 14 May 1999 (PCT/US98/22397 filed 3 Nov. 1998) in the name of Scimed Life Systems, Inc. describes devices and methods for creating a series of percutaneous myocardial revascularization channels in the heart. A catheter is described that has an outer catheter shaft that includes an anchoring shaft and a treatment shaft or probe. The anchoring shaft has at its distal end an anchor, which has a pigtail or corkscrew configuration, and can anchor the catheter to the myocardium. The treatment shaft has a distal cutting tip and extends at an angle from the distal tip of the catheter so that the treatment shaft is separated from the anchoring shaft. As the treatment shaft is located some distance away from the site where the catheter is anchored, the treatment shaft is not in stable contact with the heart while the heart is contracting. Additionally, the force required to deploy a large intramural needle is likely to bend the catheter as the anchoring shaft is located at an angle to the treatment shaft. As the anchoring and treatment shafts are located side by side within the outer lumen of the catheter, the anchoring and treatment shafts each have to be of a very small size to allow the total diameter of the catheter to be small enough to be clinically useful. The constraints imposed on the size of the treatment shaft limits the diameter of the needle and hence reduces the ablation lesion diameter.

U.S. Pat. Nos. 6,102,887 and 6,346,099 issued to Altman on 5 Aug. 2000 and 12 Feb. 2002, respectively, describe a catheter system for injecting therapeutic agents within a body tissue including the heart. A catheter includes a deployable distally penetrating structure that delivers agents within a heart wall. The penetrating structure is depicted to be a hollow helical needle for securing the delivery catheter to prevent misplacement that may result because of the motion of the beating heart. The helical needle can be screwed into the tissue prior to delivery of a drug. Another penetrating structure incorporates a solid helix, and a hollow centrally located needle may be provided. However, this system suffers from the same disadvantages described above in relation to U.S. Pat. No. 5,281,218 issued to Imran.

Thus, a need clearly exists for surgical equipment that can create thermal ablation lesions using a percutaneously or endoscopically delivered intramural needle to deliver electrical energy and that allows a user to steer a catheter to the area of interest and secure the catheter firmly to the myocardium with a fixation helix. The equipment must make efficient use of space to allow a maximum diameter needle to be deployed.

### SUMMARY

In accordance with an aspect of the invention, a surgical device for treating tissue is provided. The device comprises: a catheter; a helical fastening needle for fastening the end of the catheter to the tissue; a mechanism for deploying and retracting the helical fastening needle from and into an end of the catheter; a shaft disposed within the lumen of the catheter; a needle-like member coupled to the shaft capable of extending from the end of the catheter through the helical fastening needle into tissue and being retracting into the end of the catheter using the shaft.

The deploying and retracting mechanism comprise a shape memory alloy wire, and the helical fastening needle may be part of the shape memory alloy wire. Preferably, the shape memory alloy wire is made from a nickel-titanium alloy.

The catheter may have a second lumen, and the shape memory alloy wire may be disposed within the second lumen if the helical fastening needle is retracted. The deploying and retracting mechanism may comprise another catheter of smaller diameter coupled to the catheter, the shape memory alloy wire disposed within a lumen of the other catheter.

Alternatively, the deploying and retracting mechanism may comprise another catheter of small diameter disposed within the lumen of the outer catheter, the helical fastening needle coupled to the other catheter capable of rotation about a longitudinal axis of the other catheter. The needle-like member and the shaft may be disposed in the lumen of the other catheter.

The needle-like member may be hollow and capable of delivering a liquid to irrigate the needle-like member.

The needle-like member may comprise an electrode for delivering electromagnetic energy to thermally ablate tissue.

The needle-like member may comprise a mechanism for measuring the temperature of at least a portion of the needle-like member. The needle-like member may comprise a mechanism for measuring electrical activity from and pacing nearby tissue through multiple ring-like electrodes attached to the exterior of the needle-like member.

In accordance with another aspect of the invention, a method for surgically treating tissue is provided. The method comprises: positioning a catheter adjacent to the tissue, the catheter comprising a helical fastening needle for fastening the end of the catheter to tissue; deploying the helical fastening needle from the catheter into tissue to fasten the catheter to tissue; extending from the end of the catheter a needle-like member coupled to a shaft through the helical fastening needle into tissue.

The method may further comprise the step of retracting the needle-like member into the end of the catheter using the shaft.

The method may further comprise the step of retracting the helical fastening needle into the end of the catheter.

The helical fastening needle may be part of the shape memory alloy wire. Preferably, the shape memory alloy wire is made from a nickel-titanium alloy. The catheter may have a second lumen, the shape memory alloy wire being disposed within the second lumen if the helical fastening needle is retracted. Alternatively, another catheter of smaller diameter may be coupled to the catheter, the shape memory alloy wire being disposed within a lumen of the other catheter.

Alternatively, another catheter of small diameter is disposed with a lumen of the outer catheter, the helical fastening needle coupled to the other catheter capable of rotation about a longitudinal axis of the other catheter. The needle-like member and the shaft may be disposed in the lumen of the other catheter.

The needle-like member may be hollow, and the method may further comprise the step of delivering a liquid using the needle-like member to irrigate the needle-like member.

The method may further comprise the step of delivering electromagnetic energy using the needle-like member to thermally ablate tissue. The method may further comprise the step of measuring the temperature of at least a portion of the needle-like member. The method may further comprise the step of measuring electrical activity from and pacing nearby tissue through multiple ring-like electrodes attached to the exterior of the needle-like member.

In accordance with still another aspect of the invention, a surgical device for treating tissue is provided. The device comprises: an outer elongate member with a lumen formed therethrough; an inner elongate member with a lumen formed therethrough, the inner elongate member disposed within the lumen of the outer elongate member and capable of rotation about a longitudinal axis of the inner elongate member; a helical fixing member coupled at a distal end of the inner elongate member capable of extending from and retracting into the outer elongate member for screw-in type engagement with the tissue to connect a distal end of the outer elongate member with the tissue; and a needle-like member disposed within a portion of the lumen of the inner elongate member capable of being extended from and retracted into an end of the elongate member, the needle-like member capable of being extended concentrically through the helical fixing member into the tissue.

The outer and inner elongate members may each be a catheter. Preferably, the needle-like member is hollow and is capable of delivering a liquid to irrigate the electrode tissue interface. The needle-like member may be an electrode. Alternatively, the needle-like member may have one or more ring-like electrodes disposed circumferentially about the needle-like member. The device may comprise a conductor passing through the lumen of the inner elongate member and connected with the needle-like member for delivering the electromagnetic energy to an electrode(s) for thermal ablation.

The helical fixing member may be made of metal.

The needle-like member may further comprise means for measuring the temperature of at least a portion of the needle-like member. Still further, the needle-like member may comprise means for measuring electrical activity from and pacing the nearby tissue through multiple ring-like electrodes attached to the exterior of the needle-like member.

The device may comprise an irrigation tube located within the needle-like member, wherein the needle-like member has at least one bore for releasing irrigation liquid. Further, the device may comprise an ultrasound sensing device located within the needle-like member.

The device may further comprise a valve between the outer and inner elongate members, and a valve between the inner elongate member and the needle-like member. The device may further comprise a pull wire connected to a metal ring located at the distal portion of the catheter enabling the catheter to be flexed and deflexed as required.

In accordance with a further aspect of the invention, a surgical method for treating tissue is provided. The method comprises the steps of: positioning an outer elongate member with a lumen formed therethrough adjacent to the tissue to be treated; providing an inner elongate member with a lumen formed therethrough, the inner elongate member disposed within the lumen of the outer elongate member and capable of rotation about a longitudinal axis of the inner elongate member; twisting a helical fixing member coupled at a distal end of the inner elongate member capable of extending from and retracting into the outer elongate member for screw-in type engagement with the tissue to connect a distal end of the outer elongate member with the tissue for engagement with the tissue; and deploying into the tissue a needle-like member disposed within a portion of the lumen of the inner elongate member capable of being extended from and retracted into an end of the elongate member, the needle-like member capable of being extended concentrically through the helical fixing member into the tissue.

The outer and inner elongate members may each be a catheter. Preferably, the needle-like member is hollow and is capable of delivering a liquid to irrigate the electrode tissue interface. The needle-like member may be an electrode. Alternatively, the needle-like member may have one or more ring-like electrodes disposed circumferentially about the needle-like member.

The method may further comprise the step of delivering the liquid via the needle-like electrode to irrigate the tissue.

The helical fixing member may be made of metal.

Preferably, the tissue is located in the heart or another organ that can be reached through the vasculature.

The method may further comprise the step of measuring the temperature of at least a portion of the needle-like member.

Preferably, a valve is provided between the outer and inner elongate members, and a valve is provided between the inner elongate member and the needle-like member.

Preferably, a pull wire connected to a distal metal ring is provided.

The method may further comprise the step of judging the depth that the needle-like member is to be inserted into the tissue using an ultrasound sensing device located within the needle-like member.

In accordance with a still further aspect of the invention, a surgical device for treating tissue is provided. The device comprises: an outer elongate member with a lumen formed therethrough; a shape memory alloy wire disposed within a further lumen and having a helical shape at one end if extended from the end of the outer elongate member for screw-in type engagement with the tissue to connect the end of the outer elongate member with the tissue; a needle-like member; and an inner elongate member coupled to the needle-like member disposed within the lumen, the needle-like member capable of being extended from an end of the outer elongate member concentrically through a helical portion of the shape memory alloy wire into the tissue.

The outer elongate member may be a catheter.

The inner elongate member may be a catheter.

The needle-like member may be hollow and capable of delivering a liquid to irrigate the needle-like member. A conductor may be passed through the lumen of the inner elongate member and connected with an electrode of the needle-like member for delivering electromagnetic energy for thermal ablation. The needle-like member further may comprise a device for measuring the temperature of at least a portion of the needle-like member.

The needle-like member further may comprise a device for measuring electrical activity from and pacing the nearby tissue through multiple ring-like electrodes attached to the exterior of the needle-like member.

The device may comprise an irrigation tube located within the needle-like member; wherein the needle-like member has at least one outlet hole for releasing irrigation liquid.

The device may comprise an ultrasound sensing device located within the needle-like member.

The device may comprise a valve between the outer and inner elongate members. The device may comprise a valve between the inner elongate member and the needle-like member.

The device may comprise a pull wire connected to a metal ring attached to the distal portion of the outer elongate member.

The needle-like member may have an outer adjacent an end of the needle-like member for delivering a substance to the tissue.

The device may comprise a plurality of temperature sensing or measuring devices attached to the needle-like member and arranged at intervals to enable sensing or monitoring of temperature at a plurality of tissue depths.

The outer elongate member may be formed by extruding to provide the lumen and the further lumen.

The device may comprise another elongate member attached to the outer elongate member, the other elongate member having the further lumen.

Preferably, the shape memory alloy wire is made of a nickel-titanium alloy.

In accordance with another aspect of the invention, a surgical method for treating tissue. The method comprises the steps of: positioning an outer elongate member with a lumen formed therethrough adjacent the tissue; extending a shape member alloy wire disposed within a further lumen and having a helical shape at one end if extended from the end of the outer elongate member for screw-in type engagement with the tissue to connect the end of the outer elongate member with the tissue; and deploying into the tissue a needle-like member coupled to an inner elongate member disposed within the lumen, the needle-like member capable of being extended from an end of the outer elongate member concentrically through a helical portion of the shape memory alloy wire into the tissue.

The outer elongate member may be a catheter.

The inner elongate member may be a catheter.

The needle-like member may be hollow, and the method may further comprise the step of delivering a liquid to irrigate the needle-like member.

A conductor may be passed through the lumen of the inner elongate member and connected with an electrode of the needle-like member, and the method may further comprise the step of delivering electromagnetic energy for thermal ablation via the electrode.

The method may further comprise the step of measuring the temperature of at least a portion of the needle-like member.

The method may further comprise the step of measuring electrical activity from and pacing the nearby tissue through multiple ring-like electrodes attached to the exterior of the needle-like member.

An irrigation tube may be located within the needle-like member, the needle-like member having at least one outlet hole for releasing irrigation liquid.

An ultrasound sensing device may be located within the needle-like member.

The needle-like member may have an outlet adjacent an end of the needle-like member, and the method may further comprise the step of delivering a substance to the tissue via the outlet.

Temperature sensing or measuring devices may be attached the needle-like member and arranged at intervals, and the method may further comprise the step of sensing or monitoring temperature at a plurality of tissue depths using the plurality of temperature sensing or measuring devices.

The outer elongate member may be formed by extruding to provide the lumen and the further lumen.

Another elongate member may be attached to the outer elongate member, the other elongate member having the further lumen.

Preferably, the shape memory alloy wire is made of a nickel-titanium alloy.

### BRIEF DESCRIPTION OF THE DRAWINGS

A small number of embodiments are described herein after with reference to the drawings, in which:

FIG. 1 is a side elevation view of an intramural, needle-tipped catheter for treating myocardial tissue in accordance with an embodiment of the invention;

FIG. 2A is a detailed, side elevation view of the handle of the intramural, needle-tipped catheter of FIG. 1 (only a portion of the entire assembly of FIG. 1 is depicted);

FIG. 2B is a detailed, side elevation view of the handle of an intramural, needle-tipped catheter with an attached syringe that may be practiced in another embodiment of the invention (only a portion of the entire assembly is depicted);

FIGS. 3A, 3B, 3C, 3D, and 3E are detailed, side elevation views of needle tips that can be practiced with at least one of the catheters of FIGS. 1 and 2 (only a portion of the entire assembly is depicted);

FIG. 4 is a detailed, side elevation view of the needle tip of the catheter of FIGS. 1 and 3A (electrode rings not shown) with a helical fastening member deployed in myocardial tissue;

FIG. 5 is a detailed, side elevation view of the needle tip of the catheter of FIGS. 1, 3A and 4 with the helical fastening member and the needle-like electrode deployed in myocardial tissue;

FIGS. 6A-6D are schematic diagrams of the intramural, needle-tipped catheter in use for treating myocardial tissue in accordance with an embodiment of the invention;

FIGS. 7A-7C are schematic diagrams illustrating deployment of an existing catheter-based system adjacent to myocardial tissue and displacement of the catheter-based system during deployment of a needle, respectively;

FIG. 8A is a perspective view of an intramural, needle-tipped catheter for treating myocardial tissue in accordance with another embodiment of the invention;

FIG. 8B is a blow-up, perspective view of the needle tip of the catheter of FIG. 8A;

FIGS. 8C and 8D are cross-sectional views of the needle tip along the A-A line and the catheter along the B-B line of FIG. 8B;

FIGS. 9A-9D are schematic diagrams of the intramural, needle-tipped catheter of FIGS. 8A-8D in use for treating myocardial tissue;

FIGS. 10-12 illustrate alternate configurations of an outer body with a larger lumen and a smaller lumen;

FIGS. 13A, 13B, 13C, 13D, 13E, and 13F are more detailed views of the needle tips shown in FIGS. 3A, 3B, 3C, 3D, 3E, and 4, respectively;

FIGS. 14A, 14B, and 14C are side elevational, sectional view of the needle tip of a catheter with a helical fastening member retracted, being deployed, and deployed; and

FIGS. 15A and 15B are side elevational, sectional views of the needle tip of a catheter with a shape memory alloy wire retracted and deployed.

### DETAILED DESCRIPTION

Surgical devices for treating tissue, surgical methods for treating tissue, and intramural, needle-tipped catheters for treating tissue in the heart or other organs are described hereinafter. In the embodiments of the invention, a needle-like member may also be used for generating thermal lesions, removing tumors, and providing substances (e.g., stem cells suspended in a liquid) to the tissue, amongst other purposes. The description sets forth numerous specific details including catheter materials, metals used for electrodes, and the like. However, it will be apparent to those skilled in the art that in the light of this disclosure numerous specific modifications and/or substitutions may be made without departing from the scope and spirit of the invention. In other instances, details may not be expressed explicitly and have ben omitted so as not to obscure the invention.

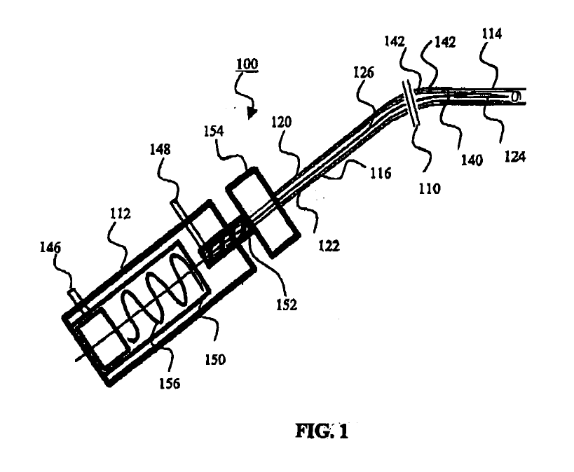

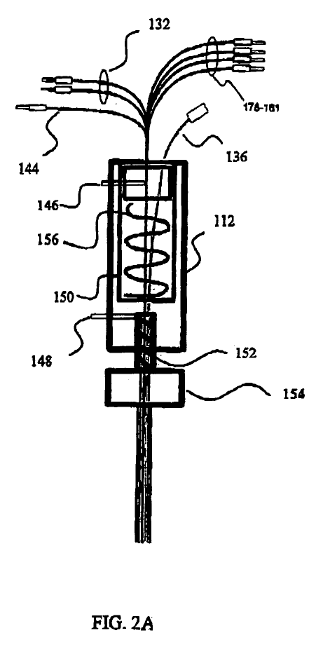

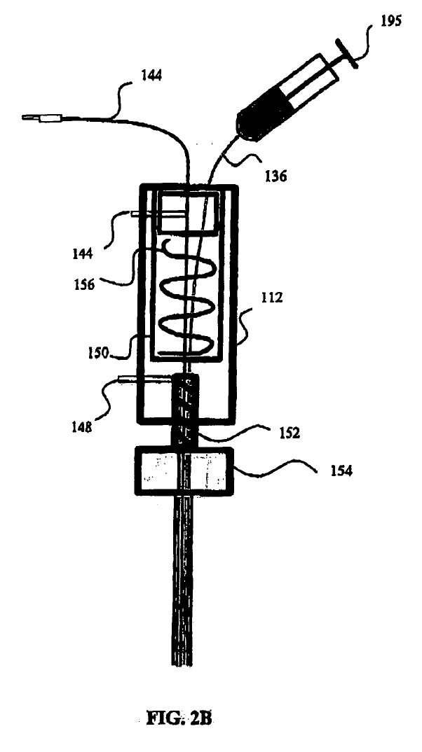

FIG. 1 is a side elevation view in cross-section of an intramural, needle-tipped device for treating myocardial tissue. The entire catheter 100 is not shown, as indicated by broken lines 110, to enable enlargement of the view of the tip of the catheter 100. FIG. 2A provides an enlarged side-elevation view of the catheter handle 112, FIG. 2B provides an enlarged side-elevation view of another catheter handle capable of receiving liquid from a syringe, and FIGS. 3A, 3B, 3C, 3D, and 3E provide enlarged, partial side-elevation view of needle tips 114. FIGS. 2A and 2B do not depict the entire assembly of the device as the catheter tip and a large portion of the catheter are omitted to emphasise the handle construction. Likewise, FIGS. 3A, 3B, 3C, 3D, and 3E do not depict the entire assembly of the device as the handle and a large portion of the catheter are omitted to emphasise the construction of the catheter tip and needle-like member. The catheter device 100 is described hereinafter with reference to FIGS. 1-3.

Generating Lesions and/or Making Measurements

With reference to FIG. 1, the catheter shaft 116 includes an outer flexible body or sheath 120 with a connection to the catheter handle 112 at one end and a distal opening at the other end. The outer body or sheath 120 is constructed of suitable material such as plastic, polyurethane, polyester or PEBAX™. A second flexible tube or torque sheath 122 is located within the outer body 120. The inner tube 122 is connected to the catheter handle 112 at one end and a helical fixing member or fastening needle 130 at the other end, as shown in FIGS. 3A to 3E. The inner tube 122 is constructed of a suitable material, such as plastic, polyurethane, polyester or PEBAX™, that allows the tube 122 to be flexible to deformation but still transmit torsional rotation to the helical fixing member or fastening needle 130 (for ease of description only, the helical fixing member or fastening needle is simply referred to as the “helical fastening needle” 130). The helical fastening needle 130 is connected to the tube 122 by an adhesive preferably.

The helical fastening needle 130 has a sharp distal tip and a length of 2 mm. However, it will be appreciated by those skilled in the art that helical fastening needles or fixing members of different sizes may be practiced without departing from the scope and spirit of the invention. The helical fastening needle 130 is made of a suitable material of fixing or fastening with tissue, such as stainless steel. The inner tube 122 and the attached helical fastening needle 130 are withdrawn or retracted into the outer body 120 so that the tube 122 and needle 130 are completely covered. An intramural needle-like member 124 can be located completely within the inner tube 122.

The intramural needle-like member 124 has a connection to a needle shaft 126 of FIG. 1, which extends back to the catheter handle 112. As shown in FIG. 2A, the needle shaft 126 in an embodiment of the invention may be coupled to an ablation wire 144 passing through the handle 112 for providing electromagnetic energy to the needle-like member 124 for thermal ablation of tissue. The electromagnetic energy may include radiofrequency (RF), microwave or ultrasound energy. The ablation wire 144 may terminate in a 2 mm plug to enable the wire to be connected to a electromagnetic energy generator, e.g. a standard RF current generator. The needle-like member 124 may itself be an electrode for delivery of energy or have one or more electrodes in or on the needle-like member 124 for delivery of energy.

The needle-like member 124 has an inner lumen and a sharp distal tip to allow penetration of myocardial or other tissue. A temperature-sensing device may be located within the inner lumen or on the external surface of the needle-like member 124. Preferably, the temperature sensing device is a thermocouple 128, which is more preferably placed 3 mm from the distal tip of the needle-member 124. The thermocouple 128 is connected to two wires 132 that extend proximally through the catheter handle 112. The wires 132 preferably terminate in 2 mm plugs to enable temperature monitoring during thermal tissue ablation.

As shown in FIG. 3A, pressure valves 190 are located between the inner and outer tubes 122, 120 and between the inner tube 122 and the needle-like member 124. The pressure valves 190 are shaped as hollow discs preferably made of an elastic material such as rubber. The pressure valves 190 are preferably attached to the exterior surface of the inner tube 122 and the exterior surface of the needle-like member 124. The pressure valves 190 allow axial and rotational movement of the inner tube 122 and the intramural needle-like member 124, but stop fluid or blood traveling back through the inner lumens of the catheter 100.

The needle-like member 124 can be extended and retracted in a controlled manner using the twistable handle shown in FIG. 2A. Thus the depth of insertion of the needle-like member into tissue can be controlled.

As shown in FIG. 3A, ring-shaped electrodes 170-173 are preferably located on the external surface of the needle-like member 124 in one embodiment of the invention to allow electrical activity within the tissue to be recorded at different depths. These electrodes 170-173 also enable the tissue around the needle-like member 125 to be paced. The ring-shaped electrodes 170-173 are constructed of a suitable conductor, preferably metal that is crimped or glued to the external surface of the needle-like member 124. The inner surface of the ring electrodes 170-173 are coated with an electrical insulator so that the ring electrode 170-173 is electrically isolated from the needle-like member 124. As shown in FIG. 3A, each electrode 170-173 has a respective wire 174-177 that is glued to the external surface of the needle-like member 124. The wires 174-177 preferably terminate in 2 mm plugs 178-181, as shown extending from the handle in FIG. 2A, to be connected to a standard electrophysiological recording system.

As shown in FIG. 3A, a fine bore irrigation tube or channel 134 is located within the lumen of the intramural, needle-like member 124. Preferably, the irrigation tube 134 terminates 1 mm from the tip of the needle-like member 124. The other end of the irrigation tube 134 extends to the catheter handle 112 and terminates in a standard ‘luer lock’ intravenous fluid connection 136. More preferably, the needle-like member 124 includes one or more outlet holes or bores 138 to allow irrigation fluid to exit from the lumen of the needle electrode 124 and enter the circulation. In this embodiment, the outlet holes or bores 138 are located at least 5 mm proximal to the maximum insertion depth of the needle-like member so that the irrigation fluid is not expelled into the tissue being ablated. As shown in FIG. 1, a pull wire 142 is connected at one end to a metal ring 140 attached to the inside of the outer catheter body 120 and at the other end to a level 148 on the catheter handle 112. Manipulation of the lever 148 enables the distal catheter tip to be flexed and deflexed to facilitate catheter placement.

The catheter handle 112 also has a sliding retraction/extension mechanism with a lever 146 coupled to a retraction spring 156 in an inner housing 150 within the handle 112. A pivotable elongated member 152 (preferably a screw) connects a rotatable attachment or dial 154 for the catheter to the body of the handle 112.

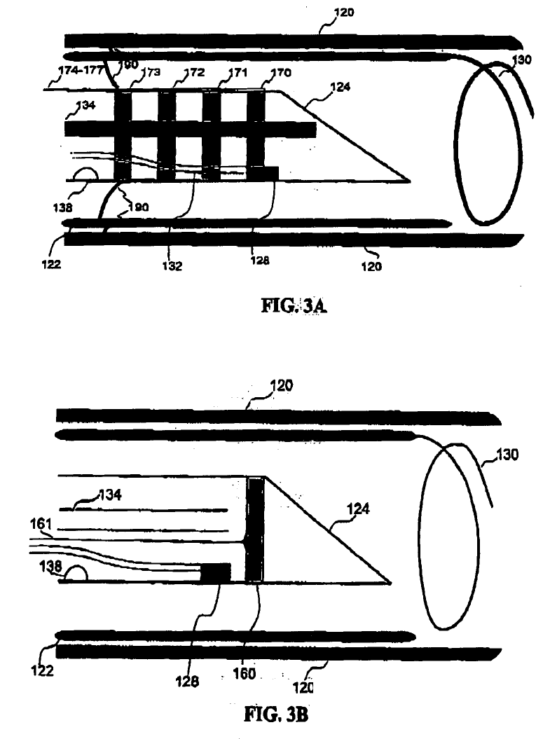

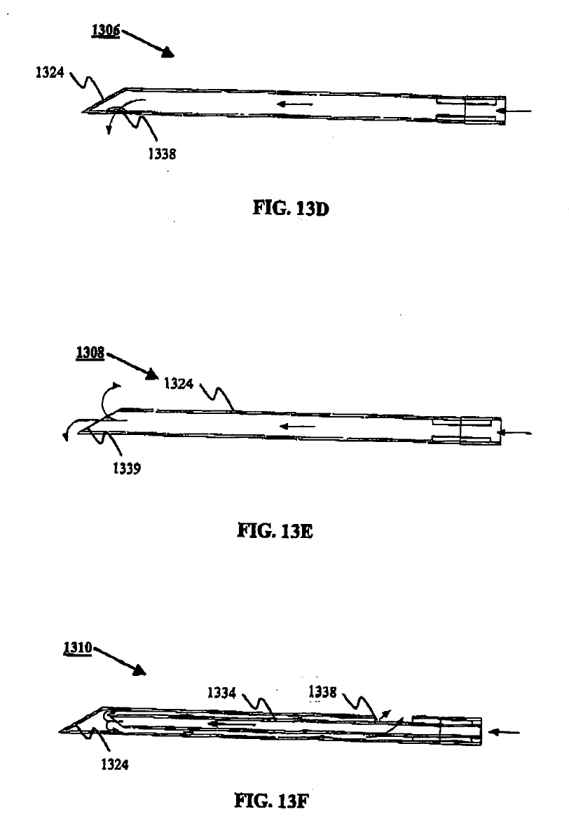

FIG. 13A is a perspective view of the needle-like member 124 of FIG. 3A, showing its configuration 1300 in detail. Corresponding references numerals 13XX, are used in FIGS. 13A-13F for those of FIGS. 13A-13E and 4. As shown in FIG. 13A, there are four ring-like electrodes 1370-1373 disposed around the needle 1324 at given distances.

Another embodiment is shown in FIG. 3B, in which like features of FIG. 3A have the same reference number. The drawing has been simplified so as not be obscure details of this embodiment. For example, while ring electrodes are not depicted in FIG. 3B, the needle 124 may be practiced with such ring electrodes. An ultrasound crystal 160 is located at the distal portion of the internal lumen of the intramural needle-like member 124. The ultrasound crystal 160 is made of a suitable piezo-electric material. The ultrasound crystal 160 has a high resonant frequency, preferably greater than 10 mega Hertz to ensure that the crystal 160 is of minimal thickness. The ultrasound crystal 160 has a pair of conductive wires 161 connected to the crystal 160 that extend back to the catheter handle 112. The conductive wires 161 are connected to an ultrasound pulser/receiver to enable A mode images to be displayed from the crystal 160.

During deployment of the intramural needle-like member 124 pulses of ultrasound energy are transmitted and received by the crystal 160. A suitable display instrument such as an oscilloscope can display the information received from the ultrasound crystal 160. For example, the thickness of the myocardium that the needle-like member 14 is in contact with can be measured from the oscilloscope display, because the epicardial surface of the heart is seen as an area of high ultrasound reflectivity. This improves the safety of the technique by allowing the operator to judge the depth that the needle-like member 124 should be inserted into the tissue. By avoiding over insertion of the needle-like member the risk of complications such as myocardial rupture, cardiac tamponade, and damage to the epicardial coronary arteries can be minimised in the given example.

FIG. 13B is a lengthwise, cross-sectional view of a needle-like member like the one of FIG. 3B, showing its configuration 1302 in detail. An ultrasound crystal 1360 is disposed at the needle tip 1324 and has conductive wires that can be connected to an ultrasound pulser/receiver. A first bore irrigation tube or channel 1334 is located within the lumen of the intramural needle-like member 1324. Fluid delivered via the irrigation tube 1334 can exit the needle 1324 via the port 1338 located rearwardly relative to the needle tip.

A further embodiment is shown in FIG. 3C in which the drawing has again be simplified so as not to obscure the details of this embodiment. Multiple small temperature sensing or measuring devices 194-197, preferably thermocouples, are attached to the outer surface of the needle-like member 124. The temperature measuring devices 194-197 are spaced at regular intervals, preferably 2 to 5 mm. Sensing wires 193 can connect the temperature measuring devices 194-197 to a temperature analysis system. The temperature sensing devices 194-197 enable the operator to monitor the temperature response at a variety of tissue depths. During the ablation procedure, the temperature measuring devices 194-197 located within the tissue (e.g., myocardium) show a temperature rise. The temperature measuring devices 194-197 that are outside of the tissue (e.g., myocardium) do not show a temperature rise during ablation as those temperature measuring devices 194-197 are cooled by the circulation of fluid (e.g., blood) past the devices. This results in increased efficiency of the ablation procedure as the operator knows the exact depth of needle insertion by observing at which points in the needle-like member 124 the temperature increases during ablation.

FIG. 13C is a lengthwise, cross-sectional view of a needle-like member like the one of FIG. 3C, showing its configuration 1304 in detail. Again, a fine bore irrigation tube or channel 1334 is located within the lumen of the intramural needle-like member 1324. Fluid delivered via the irrigation tube 1334 can exit the needle 1324 via the port 1338 located rearwardly relative to the needle tip. The needle-like member 1324 has multiple small temperature sensing or measuring devices 1394-1397, preferably thermocouples, attached to its outer surface. The temperature measuring devices 1394-1397 are spaced at regular intervals. Sensing wires inside the catheter can connect to the temperature measuring devices 1394-1397 through the needle to a temperature analysis system.

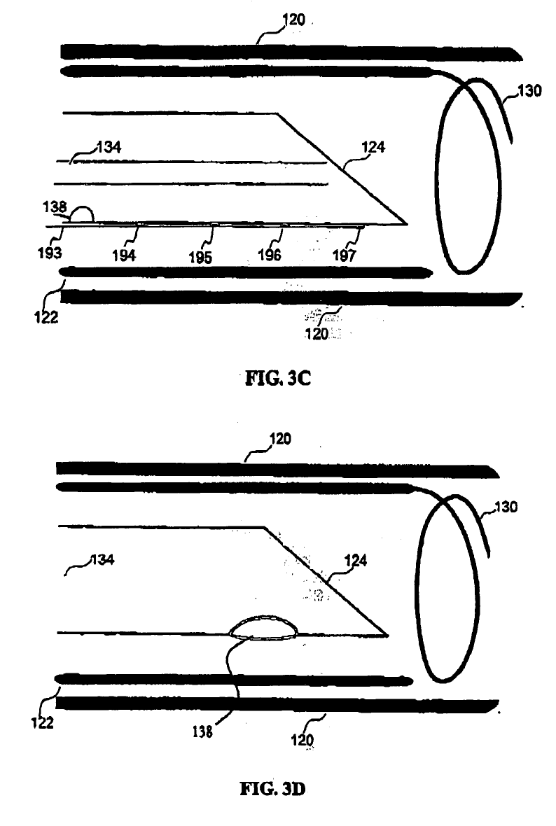

Once the end of the catheter 100 has been steered to the area of interest, the operator rotates the dial 154 on the handle 112. This rotation is transmitted to the inner torque sheath 122, advancing the helical fastening needle 130 up to 2 mm into the tissue (e.g., myocardium) 199 as shown in FIG. 4. After the helical fastening needle 130 has fixed the end of the catheter 100 against the tissue (e.g., myocardium), the operator can then advance the ablation needle electrode 124 into the tissue 199 to the required depth as shown in FIG. 5. Electromagnetic energy (e.g. electrical current for RF energy) can then be delivered to the ablation needle-like member 124 and irrigation fluid can be circulated through the ablation needle-like member 124 at a suitable rate (e.g., 20 ml/minute).

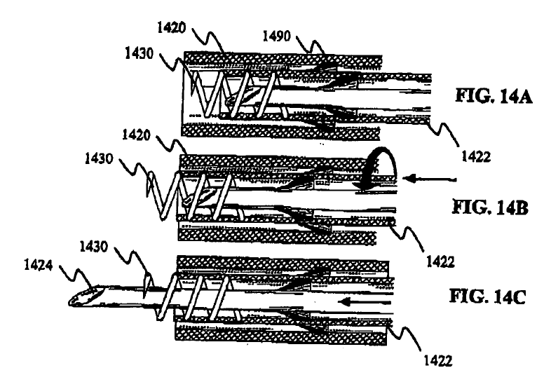

FIGS. 14A, 14B, and 14C are detailed views showing the deployment of a helical fastening needle 1430, followed by deployment of the ablation needle member 1424. As shown in FIG. 14A, the helical fastening needle 1430 and the needle member 1424 are initially retracted within the lumen of the outer catheter 1420. The helical fastening needle 1430 is disposed partially within the lumen of an inner catheter 1422, but projects forwardly from that catheter 1422 toward the opening of the outer catheter 1420. The needle member 1424 is retracted within the lumen of the inner catheter 1422. Pressure valves 1490 are disposed between the inner surface of the lumen of the outer catheter 1420 and the outer surface of the inner catheter 1422, and between the inner surface of the inner catheter 1422 and the needle member 1424. In FIG. 14B, the inner catheter 1422 is rotated (as indicated by an arrow) as the catheter 1422 is pushed forward so that the helical fastening needle 1430 protrudes from the outer catheter 1420. In the presence of tissue, this would cause the helical fastening needle to engage such tissue and attach the end of the catheter to the tissue. In FIG. 14B, the needle member 1424 and pushed forward within the outer catheter 1420. In FIG. 14C, the needle member 1424 deploys through the helical fastening needle 1430 and extends beyond that needle 1430.

FIG. 13F is a lengthwise, cross-sectional view of a needle-like member like the one of FIG. 4, showing its configuration 1310 in detail. The needle-tip member 1324 has an internal irrigation tube 1334 to deliver coolant, for example, which exits the needle by the rearward port 1338.

As depicted in FIG. 4, the helical fastening needle 130 extends preferably only up to 2 mm into the tissue (e.g., myocardium), while the needle-like member preferably extends up to 12 mm into the tissue. Other size needle-like members and helical fastening needles may be practiced without departing from the scope and spirit of the invention. The irrigation fluid exits the needle-like member through the port 138 and may enter the surrounding fluid through the end of the catheter 100 abutting the tissue (e.g., myocardium).

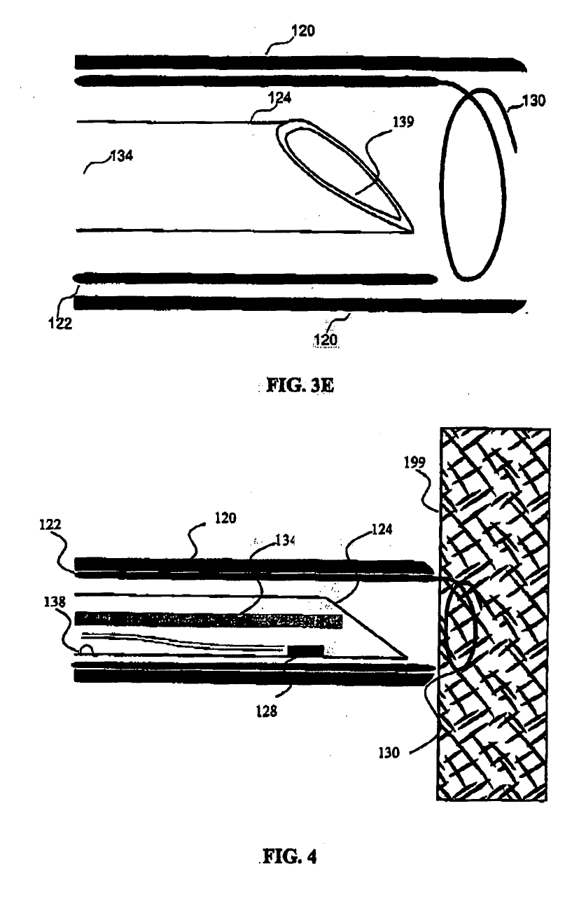

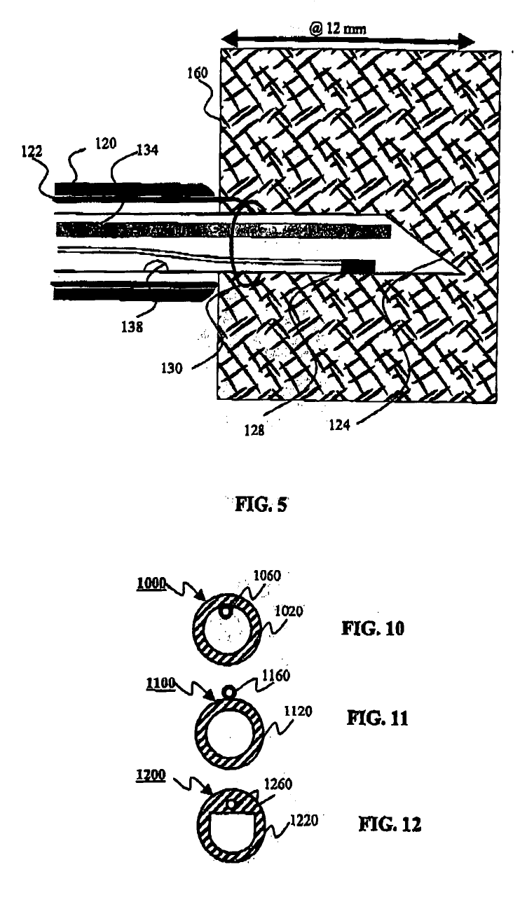

Fixation of the end of the catheter 100 against the tissue 199 enables the ablation needle-like member 124 to be inserted into the tissue 199 easily and to be directed into the tissue at the correct angle. This is done by advancing or withdrawing the catheter 100 with the helical fastening needle 130 partially deployed. This technique or process is illustrated by FIGS. 6A-6D of the drawings.

Delivering Substances to Tissue

FIG. 2B is an alternate embodiment of the catheter handle shown in FIG. 2A. Features with the same reference numeral in FIG. 2B correspond to the like features in FIG. 2A, and the corresponding description has not been duplicated to avoid repetition. In FIG. 2B, a syringe 195 is coupled to the ‘luer lock’ intravenous fluid connection 136. In this manner, substances may be delivered to tissue using the needle-like members in accordance with the embodiments depicted in FIGS. 3D and 3E. For example, stem cells may delivered in a solution from the syringe 195 via the needle-like member to the tissue.

FIG. 3D depicts a needle-like member having a closed terminal end (as per FIGS. 3A, 3B, and 3C) but with an outlet bore or hole 138 adjacent to the end of the needle-like member 124 to deliver the substance into tissue.

FIG. 13D is a lengthwise, cross-sectional view of a needle-like member like the one of FIG. 3D, showing its configuration 1306 in detail. The needle-like member 1324 has a closed terminal end, with an outlet bore or hole 1338 adjacent the end of the needle-like member 1324 to deliver the substance into tissue. For example, active substances can be delivered via the port 1338 in the side of the needle.

FIG. 3E depicts a needle-like member having an open terminal end 139 to deliver the substance into tissue in accordance with another embodiment of the invention. FIG. 13E is a lengthwise, cross-sectional view of a needle-like member like the one of FIG. 3E, showing its configuration 1308 in detail. The needle-like member 1324 has an open terminal end 1339, which may be used to deliver active substances, for example.

Deployment of Device

FIG. 6A illustrates schematically a portion 620 of a catheter 600 of the type 100 depicted in FIGS. 1-5 that is deployed near the area of interest 650 in myocardial tissue 610. The catheter tip contains a helical fastening needle 630 and a needle-like member 640 retracted within the catheter and the tip is orientated obliquely to the ventricular wall.

FIG. 6B illustrates the helical fastening needle 630 advanced one turn allowing the helical fastening needle 630 to fix the catheter to the tissue (e.g., myocardium) 610 at one point. Further manipulation of the catheter 600 causes the catheter 600 to pivot at the fixed point. The operator advances the catheter 600 (as indicated by an arrow in FIG. 6C) to orientate the catheter tip perpendicularly to the ventricular wall, as shown in FIG. 6C.

FIG. 6D illustrates the intramural needle-like member 640 extended perpendicularly into the tissue 610 to fully cover the area of interest 650 (not shown in FIGS. 6B and 6D to simplify the drawings).

Preferably, the ablation needle-like member 124, 640 is irrigated with the irrigation fluid, which is then channelled into the circulation. This is done at a portal 138 at a distance away from the tip of the needle needle-like member 124, 640. If irrigation fluid were to be expelled from the tip of the needle, the irrigation fluid would be forced under pressure into the tissue (e.g., myocardium) 199, 610, leading to local swelling. Only small quantities of irrigation fluid could be delivered if the fluid were to be expelled into the tissue 199, 610 thereby limiting the ability of the irrigation fluid to effectively cool the ablation electrode 124, 640. Thus, the positioning of one or more portals 138 away from the portion of the needle to be placed in myocardial tissue is advantageous.

Further Embodiments

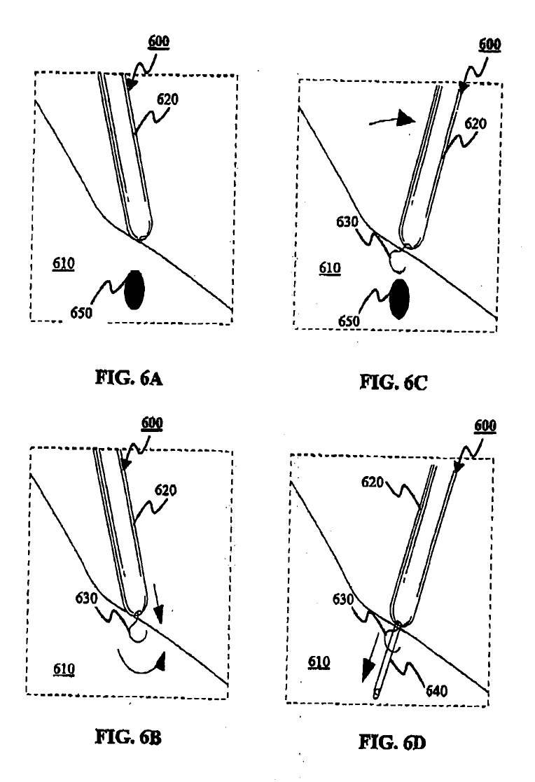

FIG. 8A is a perspective view of an intramural, needle-tipped device for treating myocardial tissue in accordance with another embodiment of the invention. Again, the entire catheter 800 is not shown, as indicated by broken lines 810, to enable enlargement of the view of the tip of the catheter 100. FIG. 8A provides an enlarged perspective view 890 of the needle tip.

The catheter shaft 816 includes an outer flexible body or sheath 820 with a connection to the catheter handle 812 at one end and a distal opening at the other end. The outer body or sheath 820 is constructed of suitable material such as plastic, polyurethane, polyester or PEBAX™. The outer body 820 has a large lumen and a smaller lumen 860, as depicted in FIGS. 8B and 8D. The smaller lumen 860 may be formed in the catheter wall defining the larger lumen. Within the smaller lumen 860, a helical fixing member or fastening needle 830 constructed of a shape memory alloy wire may be deployed at the end of catheter. The helical fixing member is connected to the catheter handle 812 and is deployed by being pushed forward out of the catheter. Once the helical fixing member has exited the confines of the inner lumen 860, the helical fixing member returns to its programmed helical shape as the helical fixing member enters the tissue 199.

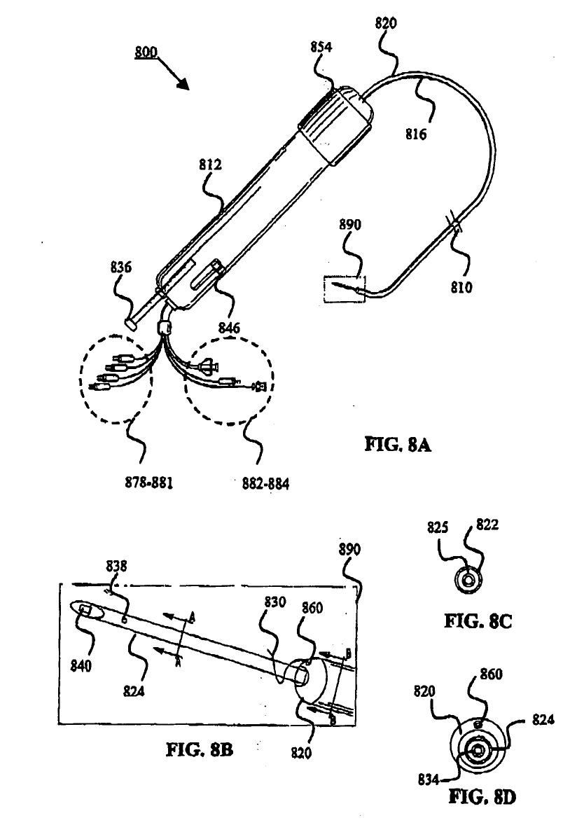

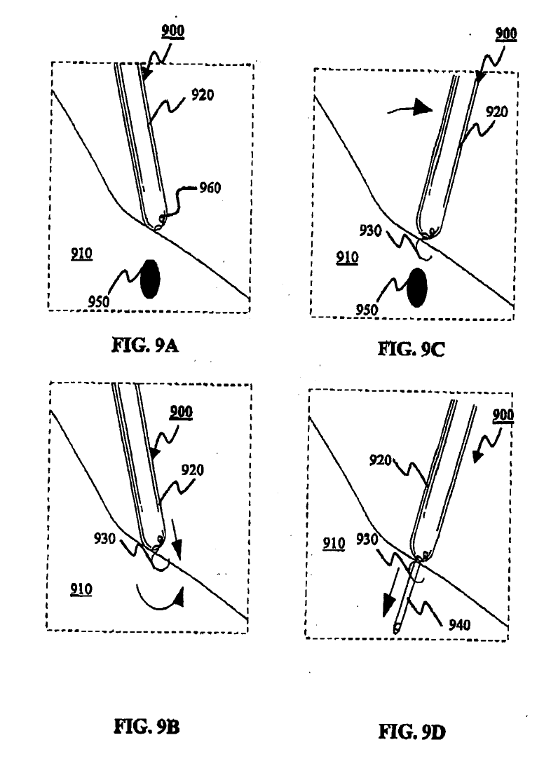

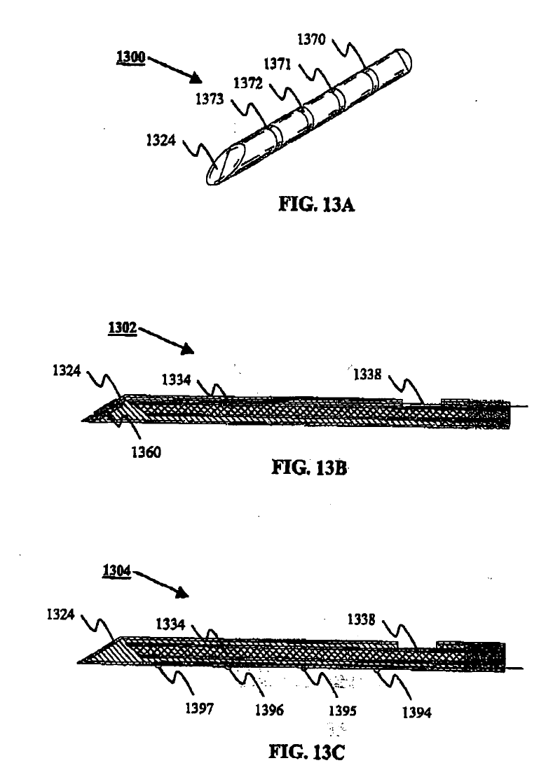

FIGS. 10-12 show alternative configurations of the outer body 820 with a larger lumen and a smaller lumen/catheter. The drawings in FIGS. 10-12 have been greatly simplified to show these configurations. In FIG. 10, the catheter 1000 has an outer body 1020, with an inner catheter 1060 attached to an internal surface of the catheter body 1020. For example, the two catheters may be extruded as a single unit, or may be separate catheters affixed together (e.g. using adhesive). This inner catheter 1060 has a lumen that is smaller than the lumen of the catheter 1000, which may have the Nitinol™ wire deployed within it. FIG. 11 shows a related configuration where the catheter has the smaller diameter catheter 1160 deployed on the outer surface of the catheter body 1120. Still further, FIG. 12 shows another configuration akin to that in FIG. 8D, but where the outer body 1220 of the catheter 1200 has a flat surface, which thickens a portion f the catheter wall. In the thickened portion of the outer body 1220 is formed the smaller lumen in which the Nitinol wire may be deployed.

The helical fastening needle 830 in this embodiment comprises a helically shaped nickel-titanium alloy, such as Nitinol™, wire. The needle 830 is shaped by heating the wire, forming the helical shape, and then cooling the wire. If retracted into the smaller lumen 860, the wire has a substantially straight shape. However, as the wire is deployed out of the smaller lumen 860 of the outer body 820, the wire resumes its helical shape as the fastening member 830. While specific materials are recited in this embodiment for the wire, many other shaped memory materials may be practiced without departing from the scope and spirit of the invention, provided they provide similar functionality and safety concerns. The helical fastening needle 830 is connected to the tube by an adhesive preferably. The helical fastening needle 830 has a sharp distal tip.

The helical fixing member 830 may be completely withdrawn into the catheter so that the helical fixing member 830 does not damage any internal organs while the catheter is being manipulated. An intramural needle-like member 824 shown in FIGS. 8B, 8D, and 8D can be located completely within the lumen of the outer body 820. The catheter 800 of FIGS. 8A-8D may be practiced with any of the needles shown in FIGS. 3A-3E.

The intramural needle-like member 824 has a connected to a needle shaft (not shown), which extends back to the catheter handle 812 of FIG. 8A. Again, the needle shaft may be coupled to an ablation wire passing through the handle 812 for providing electromagnetic energy to the needle-like member 824 for thermal ablation of tissue in the manner described above. Again, the needle-like member 824 may itself be an electrode for delivery of energy or have one or more electrodes in or on the needle-like member 824 for delivery of energy.

The needle-like member 824 has an inner lumen and a sharp distal tip to allow penetration of myocardial or other tissue. An ultrasound crystal 840 may be located at the tip of the needle-like member 824. A temperature-sensing device 838 may be located within the inner lumen or on the external surface of the needle-like member 824, as shown in FIG. 8b. Preferably the temperature-sensing device is a thermocouple. The thermocouple may be connected to two wires that extend proximally through the catheter handle 812. The needle-like member 824 can be extended and retracted in a controlled manner using the twistable handle shown in FIG. 8A. Thus the depth of insertion of the needle-like member 824 into tissue can be controlled.

While not shown in FIGS. 8A-8D, ring-shaped electrodes may be located on the external surface of the needle-like member 824 to allow electrical activity within the tissue to be recorded at different depths. These electrodes may be constructed and used in the manner noted above. Each electrode may have a respective wire terminating in a plug 878-881 to be connected to a standard electrophysiological recording system. Also depicted in FIG. 8A are a Luer lock connector 882 for irrigation fluid, a 2 mm tip electrode for connection an RF generator, and a BNC connection 884 to link the ultrasound crystal 840 to a suitable display device.

As shown in FIG. 8D, a fine bore irrigation tube or channel 834 may be located within the lumen of the intramural needle-like member 824. Preferably, the irrigation tube 834 terminates near the tip of the needle-like member 824. The other end of the irrigation tube 834 extends to the catheter handle 812 and terminates in a standard ‘luer lock’ intravenous fluid connection 882. More preferably, the needle-like member 824 includes one or more outlet holes or bores 838 to allow irrigation fluid to exit from the lumen of the needle electrode 824 and enter the circulation. A pull wire may be connected at one end of the outer catheter body 820 and at the other end to a lever on the catheter handle. As noted above, manipulation of the lever enables the distal catheter tip to be flexed and deflexed to facilitate catheter placement.

The catheter handle 812 also has a sliding retraction/extension mechanism for the distal ablation needle 824 with a lever 826 coupled to a retraction spring in an inner housing within the handle 812. The helical fixing member 830 is extended into the tissue by manipulation of the lever 836 on the catheter handle 812. After the helical fastening needle 830 has fixed the end of the catheter 800 against the tissue (e.g., myocardium), the operator can then advance the ablation needle electrode 824 through the helical fastening needle 830 into the tissue. Electromagnetic energy (e.g. electrical current for RF energy) may then be delivered to the ablation needle-like member 824. During RF ablation irrigation fluid may be delivered via a small diameter irrigation tube 834 and circulated through the ablation needle-like member 824 at a suitable rate (e.g., 20 ml/minute).

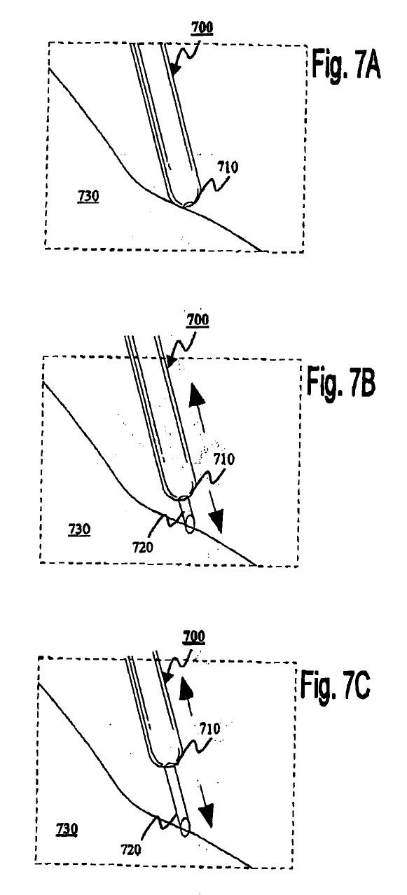

Fixation of the end of the catheter 800 against the tissue enables the ablation needle-like member 824 to be inserted into the tissue easily and to be directed into the tissue at the correction angle. This is done by advancing or withdrawing the catheter 800 with the helical fastening needle 830 partially deployed. This technique or process is illustrated by FIGS. 9A-9D of the drawings.

Deployment of Device

FIGS. 9A-9D shown the deployment of the catheter device 900. This device 900 has the construction of the catheter device 800 shown in FIGS. 8A-8D. FIGS. 6A-6D are essentially identical as those of FIGS. 9A-9D, with the elements of 6XX in FIG. 6 having like number 9XX in FIG. 9 (e.g., tissue 610 in FIG. 6 and tissue 910 in FIG. 9), except that the helical fastening member 930 deploys from the smaller lumen 960, rather than the larger lumen of FIGS. 6 and 9. For the sake of brevity, the details of FIG. 6 are not repeated.

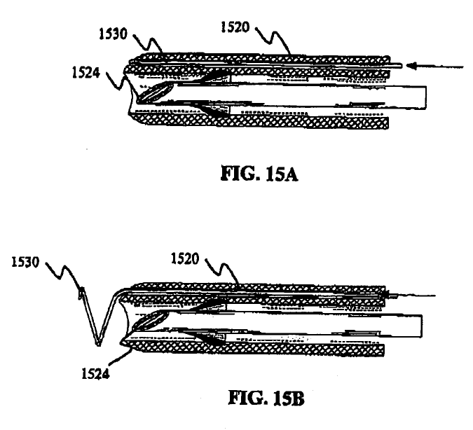

FIGS. 15A and 15B are detailed views showing the deployment of shape memory alloy wire as a helical fastening needle 1530, followed by deployment of the ablation needle member 1524. As shown in FIG. 15A, the shape memory alloy wire 1430 and the needle member 1524 are initially retracted within respective lumens of the catheter 1520. The shape memory alloy wire 1530 is disposed within its own lumen in this embodiment and is substantially straight as retracted. The needle member 1524 is retracted within the lumen of the catheter 1520. A pressure valve 1590 is disposed between the inner surface of the large lumen of the catheter 1520 and the needle member 1524. In FIG. 15B, the shape memory alloy wire 1530 is pushed forward so that the shape memory alloy wire 1430 forms a helix as it protrudes from the catheter 1520. In the presence of tissue, this would cause the terminal helical portion of the shape memory alloy wire to engage such tissue and attach the end of the catheter to the tissue.

The embodiments of the invention have a number of advantages including the following. The embodiment of the invention enable fixation of the catheter to the tissue with a helical fastening needle. This is advantageous because the helical fastening needle can be moved independently of ablation needle-like member movement. This means that the helical fastening needle needs to be advanced only a few millimeters into the tissue to provide sufficient stabilisation for needle insertion. This is in marked contrast to catheters having a screw needle electrode for both fixation and ablation, which require the screw needle electrode to be inserted to a much greater depth. In the event of traumatic movement of the catheter (e.g., during defibrillation) the helical fastening needle of the embodiments of the invention cause negligible damage to the tissue if dislodged since the helical fastening needle is only inserted 1-2 mm. Inserting a screw electrode as deep as the required ablation can disadvantageously lead to a large myocardial tear in the event of sudden movement of the screwed-in catheter. Thus, the embodiments of the invention have improved safety.

Further, the embodiments of the invention are advantageous in that the helical fastening needle can have a small outer diameter to enable it to enter the tissue (e.g., myocardium) with minimal resistance, relative to the larger diameter of screw electrodes that are required for ablation. This provides improved ease of use.

The embodiments of the invention are useful in a number of ways. Firstly, the embodiments can be used for ablation of ventricular tachycardia originating from intramyocardial and subepicardial sites. Preliminary testing indicates that the percutaneous needle ablation catheter can create lesions of>12 mm of depth. Still further, the embodiments of the invention can be used for ablation of certain supraventricular arrhythmias where conventional ablation strategies have failed (eg atrial flutter). Still further, the embodiments of the invention can be used to provide thermal ablation therapy for cardiac or other tumours. Still further, the embodiments of the invention can be used to enable percutaneous ablation of non-cardiac tissue including but not limited to hepatic, renal and pancreatic tumours.

Thus, surgical devices for treating tissue, surgical methods for treating tissue, and intramural, needle-tipped catheters for treating myocardial tissue have been described. While only a small number of embodiments have been set forth, it will be apparent to those skilled in the art that, in view of this disclosure, modifications and substitutions may be made without departing from the scope and spirit of the invention.

The claims defining the invention are as follows:

1. A surgical device for treating tissue, comprising:
a catheter;
a helical fastening needle for fastening the end of said catheter to tissue;
means for deploying and retracting said helical fastening needle from and into an end of said catheter;
a shaft disposed within a lumen of said catheter;
a needle-like member coupled to said shaft capable of extending from the end of the catheter through said helical fastening needle into tissue and being retracting into the end of said catheter using said shaft.

2. The surgical device according to claim 1, wherein said deploying and retracting means comprise a shape memory alloy wire.

3. The surgical device according to claim 2, wherein said helical fastening needle is part of said shape memory alloy wire.

4. The surgical device according to claim 2 or 3, wherein said shape memory alloy wire is made from a nickel-titanium alloy.

5. The surgical device according to claim 3, wherein said catheter has a second lumen, said shape memory alloy wire being disposed within said second lumen if said helical fastening needle is retracted.

6. The surgical device according to claim 2, wherein said deploying and retracting means comprises another catheter of smaller diameter coupled to said catheter, said shape memory alloy wire disposed within a lumen of said other catheter.

7. The surgical device according to claim 1, wherein said deploying and retracting means comprises another catheter of small diameter disposed with a lumen of said outer catheter, said helical fastening needle coupled to said other catheter capable of rotation about a longitudinal axis of said other catheter.

8. The surgical device according to claim 7, wherein said needle-like member and said shaft are disposed in the lumen of said other catheter.

9. The surgical device according to claim 1, wherein said needle-like member is hollow and is capable of delivering a liquid to irrigate the needle-like member.

10. The device according to claim 1, wherein said needle-like member comprises an electrode for delivering electromagnetic energy to thermally ablate tissue.

11. The device according to claim 10, further wherein said needle-like member comprises means for measuring the temperature of at least a portion of said needle-like member.

12. The device according to claim 10, further wherein said needle-like member comprises means for measuring electrical activity from and pacing nearby tissue through multiple ring-like electrodes attached to the exterior of said needle-like member.

13. A method for surgically treating tissue, comprising:
positioning a catheter adjacent tissue, said catheter comprising a helical fastening needle for fastening the end of said catheter to tissue;
deploying said helical fastening needle from said catheter into tissue to fasten said catheter to tissue;
extending from the end of said catheter a needle-like member coupled to a shaft through said helical fastening needle into tissue.

14. The method according to claim 13, further comprising the step of retracting said needle-like member into the end of said catheter using said shaft.

15. The method according to claim 14, further comprising the step of retracting said helical fastening needle into the end of said catheter.

16. The surgical method according to claim 13, wherein said helical fastening needle is part of said shape memory alloy wire.

17. The surgical method according to claim 16, wherein said shape memory alloy wire is made from a nickel-titanium alloy.

18. The surgical method according to claim 16, wherein said catheter has a second lumen, said shape memory alloy wire being disposed within said second lumen if said helical fastening needle is retracted.

19. The surgical method according to claim 16, wherein said catheter of small diameter is coupled to said catheter, said shape memory alloy wire being disposed within a lumen of said other catheter.

20. The surgical method according to claim 13, wherein another catheter of small diameter is disposed with a lumen of said outer catheter, said helical fastening needle coupled to said other catheter capable of rotation about a longitudinal axis of said other catheter.

21. The surgical method according to claim 20, wherein said needle-like member and said shaft are disposed in the lumen of said other catheter.

22. The surgical method according to claim 13, wherein said needle-like member is hollow and further comprising the step of delivering a liquid using said needle-like member to irrigate said needle-like member.

23. The method according to claim 13, further comprising the step of delivering electromagnetic energy using said needle-like member to thermally ablate tissue.

24. The method according to claim 23, further comprising the step of measuring the temperature of at least a portion of said needle-like member.

25. The method according to claim 23, further comprising the step of measuring electrical activity from and pacing nearby tissue through multiple ring-like electrodes attached to the exterior of said needle-like member.

26. A surgical device for treating tissue, comprising:
an outer elongate member with a lumen formed therethrough;
an inner elongate member with a lumen formed therethrough, said inner elongate member disposed within said lumen of said outer elongate member and capable of rotation about a longitudinal axis of said inner elongate member;
a helical fixing member coupled at a distal end of said inner elongate member capable of extending from and retracting into said outer elongate member for screw-in type engagement with said tissue to connect a distal end of said outer elongate member with said tissue; and
a needle-like member disposed within a portion of said lumen of said inner elongate member capable of being extended from and retracted into an end of said elongate member, said needle-like member capable of being extended concentrically through said helical fixing member into said tissue.

27. The device according to claim 26, wherein said outer elongate member is a catheter.

28. The device according to claim 26, wherein said inner elongate member is a catheter.

29. The device according to claim 26, wherein said needle-like member is hollow and is capable of delivering a liquid to irrigate the needle-like member.

30. The device according to claim 26, wherein said helical fixing member is made of metal.

31. The device according to claim 26, further comprising a conductor passing through said lumen of said inner elongate member and connected with an electrode of said needle-like member for delivering electromagnetic energy for thermal ablation.

32. The device according to claim 31, further wherein said needle-like member further comprises means for measuring the temperature of at least a portion of said needle-like member.

33. The device according to claim 31, further wherein said needle-like member further comprises means for measuring electrical activity from and pacing the nearby tissue through multiple ring-like electrodes attached to the exterior of said needle-like member.

34. The device according to claim 26, further comprising an irrigation tube located within said needle-like member, wherein said needle-like member has at least one outlet hole for releasing irrigation liquid.

35. The device according to claim 26, further comprising an ultrasound sensing device located within said needle-like member.

36. The device according to claim 26, further comprising a valve between said outer and inner elongate members.

37. The device according to claim 26, further comprising a valve between said inner elongate member and said needle-like member.

38. The device according to claim 26, further comprising a pull wire connected to a metal ring attached to the distal portion of said outer elongate member.

39. The device according to claim 26, wherein said needle-like member has an outlet adjacent an end of said needle-like member for delivering a substance to the tissue.

40. The device according to claim 26, further comprising a plurality of temperature sensing or measuring devices attached said needle-like member and arranged at intervals to enable sensing or monitoring of temperature at a plurality of tissue depths.

41. A surgical method for treating tissue, said method comprising the steps of:
positioning an outer elongate member with a lumen formed therethrough adjacent said tissue;
providing an inner elongate member with a lumen formed therethrough, said inner elongate member disposed within said lumen of said outer elongate member and capable of rotation about a longitudinal axis of said inner elongate member;
twisting a helical fixing member coupled at a distal end of said inner elongate member capable of extending from and retracting into said outer elongate member for screw-in type engagement with said tissue to connect a distal end of said outer elongate member with said tissue for engagement with said tissue; and
deploying into said tissue a needle-like member disposed within a portion of said lumen of said inner elongate member capable of being extended from and retracted into an end of said elongate member, said needle-like member capable of being extended concentrically through said helical fixing member into said tissue.

42. The method according to claim 41, wherein said outer elongate member is a catheter.

43. The method according to claim 41, wherein said inner elongate member is a catheter.

44. The method according to claim 41, wherein said needle-like member is hollow and is capable of delivering a liquid to irrigate said needle-like member.

45. The method according to claim 41, further comprising the step of delivering said liquid via said needle-like member to cool the tissue electrode interface.

46. The method according to claim 41, wherein said helical fixing member is made of metal.

47. The method according to claim 41, wherein said tissue is located in the heart or another organ that can be reached through the vasculature, a hollow organ such as the intestine or through a cavity such as but not limited to the peritoneal space or thoracic cavity.

48. The method according to claim 41, further comprising the step of delivering electromagnetic energy to said needle-like member for thermal tissue ablation via a conductor passing through said lumen and connected with an electrode of said needle-like member.

49. The method according to claim 48, further comprising the step of measuring the temperature of at least a portion of said needle-like member.

50. The method according to claim 41, wherein a valve is provided between said outer and inner elongate members.

51. The method according to claim 41, wherein a pull wire attached to a metal ring located in the distal section of said outer elongate member is provided.

52. The method according to claim 51, further comprising the step of using the pull wire to flex and deflex the outer elongate member, enabling the outer elongate member to be positioned at the region of interest.

53. The method according to claim 41, wherein a valve is provided between said inner elongate member and said needle-like member.

54. The method according to claim 41, further comprising the step of judging the depth that said needle-like member is to be inserted into said tissue using an ultrasound sensing device located within said needle-like member.

55. The method according to claim 41, wherein said positioning step involves using a pull wire attached to a distal ring to flex and deflex said outer elongate member as required.

56. The method according to claim 41, wherein said needle-like member has an outlet adjacent an end of said needle-like member for delivering a substance to the tissue.

57. The method according to claim 41, further comprising the step of sensing or monitoring temperature at a plurality of tissue depths using a plurality of temperature sensing or measuring devices attached said needle-like member and arranged at intervals.

58. A surgical device for treating tissue, comprising:
an outer elongate member with a lumen formed therethrough;
a shape memory alloy wire disposed within a further lumen and having a helical shape at one end if extended from the end of said outer elongate member for screw-in type engagement with said tissue to connect said end of said outer elongate member with said tissue;
a needle-like member; and
an inner elongate member coupled to said needle-like member disposed within said lumen, said needle-like member capable of being extended from an end of said outer elongate member concentrically through a helical portion of said shape memory alloy wire into said tissue.

59. The device according to claim 58, wherein said outer elongate member is a catheter.

60. The device according to claim 58, wherein said inner elongate member is a catheter.

61. The device according to claim 58, wherein said needle-like member is hollow and is capable of delivering a liquid to irrigate the needle-like member.

62. The device according to claim 59, further comprising a conductor passing through said lumen of said inner elongate member and connected with an electrode of said needle-like member for delivering electromagnetic energy for thermal ablation.

63. The device according to claim 62, further wherein said needle-like member further comprises means for measuring the temperature of at least a portion of said needle-like member.

64. The device according to claim 62, further wherein said needle-like member further comprises means for measuring electrical activity from and pacing the nearby tissue through multiple ring-like electrodes attached to the exterior of said needle-like member.

65. The device according to claim 58, further comprising an irrigation tube located within said needle-like member, wherein said needle-like member has at least one outlet hole for releasing irrigation liquid.

66. The device according to claim 58, further comprising an ultrasound sensing device located within said needle-like member.

67. The device according to claim 58, further comprising a valve between said outer and inner elongate members.

68. The device according to claim 58, further comprising a valve between said inner elongate member and said needle-like member.

69. The device according to claim 58, further comprising a pull wire connected to a metal ring attached to the distal portion of said outer elongate member.

70. The device according to claim 58, wherein said needle-like member has an outlet adjacent an end of said needle-like member for delivering a substance to the tissue.

71. The device according to claim 58, further comprising a plurality of temperature sensing or measuring devices attached said needle-like member and arranged at intervals to enable sensing or monitoring of temperature at a plurality of tissue depths.

72. The device according to claim 58, wherein said outer elongate member is formed by extruding to provide said lumen and said further lumen.

73. The device according to claim 58, further comprising another elongate member attached to said outer elongate member, said other elongate member having said further lumen.

74. The device according to claim 58, wherein said shape memory alloy wire is made of a nickel-titanium alloy.

75. A surgical method for treating tissue, said method comprising the steps of:
positioning an outer elongate member with a lumen formed therethrough adjacent said tissue;
extending a shape memory alloy wire disposed within a further lumen and having a helical shape at one end if extended from the end of said outer elongate member for screw-in type engagement with said tissue to connect said end of said outer elongate member with said tissue; and
deploying into said tissue a needle-like member coupled to an inner elongate member disposed within said lumen, said needle-like member capable of being extended from an end of said outer elongate member concentrically through a helical portion of said shape memory alloy wire into said tissue.

76. The method according to claim 75, wherein said outer elongate member is a catheter.

77. The method according to claim 75, wherein said inner elongate member is a catheter.

78. The method according to claim 75, wherein said needle-like member is hollow, and further comprising the step of delivering a liquid to irrigate the needle-like member.

79. The method according to claim 76, wherein a conductor passes through said lumen of said inner elongate member and is connected with an electrode of said needle-like member, and further comprising the step of delivering electromagnetic energy for thermal ablation via said electrode.

80. The method according to claim 79, further comprising the step of measuring the temperature of at least a portion of said needle-like member.

81. The method according to claim 79, further comprising the step of measuring electrical activity from and pacing the nearby tissue through multiple ring-like electrodes attached to the exterior of said needle-like member.

82. The method according to claim 75, wherein an irrigation tube is located within said needle-like member, said needle-like member having at least one outlet hole for releasing irrigation liquid.

83. The method according to claim 75, wherein an ultrasound sensing device is located within said needle-like member.

84. The method according to claim 75, wherein said needle-like member has an outlet adjacent an end of said needle-like member, and further comprising the step of delivering a substance to the tissue via said outlet.

85. The method according to claim 75, wherein a plurality of temperature sensing or measuring devices are attached said needle-like member and arranged at intervals, and further comprising the step of sensing or monitoring temperature at a plurality of tissue depths using said plurality of temperature sensing or measuring devices.

86. The method according to claim 75, wherein said outer elongate member is formed by extruding to provide said lumen and said further lumen.

87. The method according to claim 75, wherein another elongate member is attached to said outer elongate member, said other elongate member having said further lumen.

88. The method according to claim 75, wherein said shape memory alloy wire is made of a nickel-titanium alloy.
